(19) United States
(12) Patent Application Publication
(10) Pub. No.: US 2013/0315854 A1
(43) Pub. Date: Nov. 28, 2013

(54) Composition For Soft Tissue Treatment

(71) Applicant: Urogyn B.V., Nijmegen (NL)

(72) Inventor: Jan Albert de VRIES, Zelhem (NL)

(21) Appl. No.: 13/900,831

(22) Filed: May 23, 2013

(30) Foreign Application Priority Data
May 23, 2012 (NL) 2008861

(51) Int. Cl. A61L 27/18

(52) U.S. Cl. CPC A61L 27/18
USPC 424/78.08; 206/219; 604/82

(57) ABSTRACT

A composition for use in treating soft tissue in a living creature with a bulking material prepared in situ from a composition of an addition curable polysiloxane system.

### CROSS-REFERENCE TO RELATED APPLICATIONS

This application claims the benefit of NL Patent Application Serial No. 2008861, filed on 23 May 2012, the benefit of the earlier filing date of which is hereby claimed under 35 USC §119(a)-(d) and (f). This application is hereby incorporated in its entirety as if fully set forth herein.

### BACKGROUND OF THE INVENTION

1. Field of the Invention

The present invention relates generally to a composition for use in soft tissue treatment in a living creature such as a human being.

2. Description of Related Art

Soft tissue treatment, e.g. filling, augmenting, strengthening, reconstructing and expanding of soft tissue in humans is applied for various purposes.

E.g. one way of treating incontinence is described in EP-A2-1516636. This treatment comprises a step of correcting the shape of the internal urethral orifice and/or the urethra with an elastic form stable material, derived from a curable elastomer precursor composition. A patient is prepared for treatment according to standard medical procedures. Then a catheter is brought into the urethra until the injection needle reaches the bladder, e.g. indicated by the presence of a droplet of urine, and then retracted over a predetermined distance, e.g. in the range of 1-2 cm. At the position thus reached the wall of the urethra is punctured by the injection needle, and the composition is forced from the respective container via a suitable flexible tube to the needle and deposited directly adjacent this wall in the respective tissue. Preferably the composition is applied at multiple locations surrounding the urethra. A preferred composition comprises about 60-75% by weight poly(dimethyl siloxane), about 2-5% cross-linking agent, a diluent in the range of 10-20% and about 10-20% radiopaque powder, based on the weight of the entire composition. During reaction (a condensation polymerization) propanol is split off.

Gastroesophageal reflux disease (GERD) caused by malfunctioning of the lower esophageal sphincter can also be treated by injecting a suitable material into body sites surrounding the esophagus, as e.g. suggested in WO-A-03/072196 and EP-A2-1516636. In reconstructive surgery and cosmetic surgery soft tissues may also be filled using polymeric materials.

Now some drawbacks of this material known from EP-A2-1516636 have been discovered. One drawback concerns the dosing ratio of polymer:catalyst in the composition, which is in the order of 1000:1. As the starting composition is to be prepared shortly in advance of the actual application such as injection, otherwise (partial) curing would have rendered the material unworkable, in an appropriate amount typically in the ml range, the dosing amount of the catalyst is in the microliter range. Any unintended deviation affects the actual curing time and other properties, which is rather unpredictable and therefore undesired. Thus precise control of the crosslinking reaction during application is difficult to achieve. Another drawback relates to the fact that some kind of shrink occurs during crosslinking. This shrink could cause voids to be present in the cured material, resulting in a locally insufficient volume thereof. Furthermore it has appeared that the crosslinked material is susceptible to degradation over time in the presence of e.g. oxygen or oxygen containing compounds, resulting in gradually increasing brittleness. If the material becomes too brittle, fractures could be initiated and small pieces may be separated. These small pieces like any small particulates may show a tendency of migration through the body. This known material also requires storage at low temperatures (−18° C.) in a freezer in a protective atmosphere.

In yet older methods a gel like collagen is injected multiple times during treatment. Such a gel is not form stable and is degradable in the body and shows shrinkage. It has also been proposed to add non-resorbable particles to the gel. Because these particles are to be co-injected with the gel, the particle size needs to be small. However, small particles tend to migrate through the body. Therefore these older methods do not provide a long-lasting effect.

In practice the standard treatment comprises anchoring a tightened tape or strip around the urethra in the surrounding body tissue in order to counteract incontinence. However, if the tape is not positioned well, a patient cannot be treated any longer, and is deemed to suffer from incontinence always.

Therefore, it is an object of the present invention to provide a composition for use in a broad number of soft tissue treatments, such as strengthening of (muscle) tissue, in particular for treating incontinence and GERD.

Another object of the present invention is to provide a composition for such use, which is stable in time after curing.

Yet another object of the present invention is to provide a composition for such use, which is easy to compose from the individual components thereof.

A further object of the present invention is to provide a composition that does not show one or more of the drawbacks mentioned above, or at least to a lesser extent.

It is also an object to provide a composition for use in treating incontinence that has been treated insufficiently and/or ineffectively by older methods, in particular the method of applying a tape or strip.

Still another object of the present invention is to provide a method of treating soft tissue in a living creature with a stable bulking agent prepared in situ and having a long-lasting effect.

### BRIEF SUMMARY OF THE INVENTION

Briefly described, in a preferred form, the present invention comprises a composition for use in treatment of soft tissue in a living creature with bulking material prepared in situ from a composition comprising an addition curable polysiloxane system.

Surprisingly the present inventor has discovered that many of the drawbacks described above relate to the curing mechanism of the polysiloxanes. The composition disclosed in EP-A2-1516636 is crosslinked through a so called condensation cure mechanism, wherein upon crosslinking propanol is split out. This is believed to be a main cause of the shrink of the known material. Contrary to a condensation cure mechanism, a composition according to the invention is crosslinked by means of an addition cure mechanism, wherein the polymeric base, a polysiloxane, reacts with a crosslinker without generating a byproduct. Such a byproduct is considered to be responsible for shrink and degradation over time. In addition, addition curable polysiloxane compositions according to the invention can be prepared in a 1:1 ratio from the components that are generally contained in a two container dispensing system having containers of equal volume. In such a dispensing system the almost equal volumes of the starting components can be maintained separately until needed.

The starting materials of this system do not need to be stored at low temperatures. Usually the addition curable polysiloxane system can be processed up to about 10-20 minutes, e.g. 15 minutes. Thereafter curing has advanced to such an extent that the viscosity becomes too high for application, e.g. by injection. Injection is usually monitored, e.g. by X-ray imaging. The composition is less susceptible to breakdown over time (non-resorbable), as a result stability and effect are long-lasting.

The present invention also provides a prepackaged addition curable polysiloxane system for use in soft tissue treatment in a living creature with a bulking material prepared in situ from a composition comprising an addition curable polysiloxane system, comprising a two component dispensing device having two containers separated from each other by a temporary seal. In a first container at least a catalyst is contained. The other container contains at least a crosslinker. Base polysiloxane polymers may be present in both containers.

The invention further provides the use of an addition curable polysiloxane system in manufacturing a composition for use in treating soft tissue in a living creature with a bulking agent prepared in situ from said composition that comprises the addition curable polysiloxane system.

The invention also comprises a method of treating soft tissue in a living creature with a bulking agent prepared in situ from a composition comprising the addition curable polysiloxane system comprising a step of applying, in said soft tissue, an addition curable polysiloxane composition comprising a polysiloxane having an ethylenically unsaturated moiety, preferably an ethylenically unsaturated moiety terminated polysiloxane, a crosslinker and a catalyst in effective amounts for allowing an addition curing in situ, in the respective soft tissue of the living creature and a step of allowing said composition to cure.

In an exemplary embodiment, the present invention is a soft tissue treatment composition comprising an addition curable polysiloxane system. The addition curable polysiloxane system can comprise a polysiloxane, a catalyst, and a crosslinker. The addition curable polysiloxane system can further comprise one or more of a tracer material, a bactericidal agent and a filler.

The addition curable polysiloxane system can comprise a polysiloxane having an ethylenically unsaturated moiety, an ethylenically unsaturated moiety terminated polysiloxane, a vinyl terminated polysiloxane, a vinyldialkyl terminated dimethyl polysiloxane, and/or a vinyldimethyl terminated polysiloxane.

The addition curable polysiloxane system can comprise a Pt catalyst, a Pt(0) organic complex, and/or a Pt(0) vinyl siloxane complex.

The addition curable polysiloxane system can comprise a hydrosilicone crosslinker, hydrogen dialkyl terminated siloxy groups, and/or hydrogen dimethyl terminated siloxy groups.

The addition curable polysiloxane system can comprises a first container with a catalyst, and a second container with a crosslinker, wherein at least one of the first and the second container further comprise a polysiloxane.

In another exemplary embodiment, the present invention can comprise a soft tissue treatment composition comprising an addition curable polysiloxane system, wherein the addition curable polysiloxane system comprises a polysiloxane, a catalyst, a crosslinker, and optionally, one or more of a tracer material, a bactericidal agent, and a filler.

The polysiloxane can comprise an ethylenically unsaturated moiety terminated polysiloxane, the catalyst can comprise a Pt(0) vinyl siloxane complex, and the crosslinker can comprise hydrogen dimethyl terminated siloxy groups.

The tracer material can comprise titanium dioxide.

The bactericidal agent can comprise Ag⁺ zeolite.

In another exemplary embodiment, the present invention comprises a first container comprising a portion of the polysiloxane comprising a polysiloxane having an ethylenically unsaturated moiety and the catalyst, and a second container comprising a portion of the polysiloxane comprising a polysiloxane having an ethylenically unsaturated moiety, and the crosslinker. The containers can be parts of a two component dispensing device having the two containers separated from each other by a temporary seal. The two component dispensing device can further comprise a mixing means. The mixing means can comprise a static mixer and/or at least one injection needle.

In another exemplary embodiment, the present invention can comprise a method of treating soft tissue in a living creature comprising preparing a bulking agent in situ from addition curable polysiloxane, and treating the soft tissue with the bulking agent. Treating the soft tissue with the bulking agent can be selected from the group consisting of the treatment of: incontinence, reflux, reconstruction surgery, cosmetic surgery, tissue expansion, filling a body cavity caused by surgery, and prostatectomy.

Preparing the bulking agent can comprise applying the addition curable polysiloxane, applying a catalyst, applying a crosslinker, and curing the bulking agent, in situ, in proximity to the soft tissue.

The method can further comprise monitoring one or more of the preparing and treating steps. The monitoring can comprises monitoring via X-ray imaging and/or echoscopy.

Further objects of the present invention arise from the following statements and the attached claims.

### DETAILED DESCRIPTION OF THE INVENTION

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or illustrated in the drawings. The invention is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described as making up the various elements of the invention are intended to be illustrative and not restrictive. Many suitable materials that would perform the same or a similar function as the materials described herein are intended to be embraced within the scope of the invention. Such other materials not described herein can include, but are not limited to, for example, materials that are developed after the time of the development of the invention.

In this specification “soft tissue” refers to tissues that connect, support or surround other structures and organs of the body, not being bone. In this context soft tissue includes tendons, ligaments, fascia, skin, fibrous tissues, fat and synovial and mucosal membranes, and muscles, nerves and blood vessels.

The composition according to the invention is introduced in such soft tissue and cures in situ by means of a so called addition cure mechanism. Basically this mechanism involves the addition reaction of polyfunctional silicon hydride to unsaturated groups in polysiloxane chains. Although the E-modulus of the cured material is higher than the E-modulus of the known polysiloxane based materials, the cured material maintains a sufficient degree of flexibility due to the presence of relatively long chains in the cured product. This balance of properties results in a reduced risk of cracking and/or fracturing. And even, if fracturing occurs, migration is less likely.

The composition can be used in a broad spectrum of soft tissue treatments, wherein said soft tissue is not part of organs. Preferred treatments comprise treatment of incontinence, treatment of reflux, reconstruction surgery, cosmetic surgery, tissue expansion and filling a body cavity caused by surgery, in particular after prostatectomy. Incontinence treatment is applicable to both urinary and fecal incontinence, e.g. stress-induced incontinence. Examples of reflux treatment comprise GERD, renal reflux and others. Reconstructive and cosmetic surgery using the composition according to the invention can be performed at a number of body parts including ear, nose, eyelid, nipple, breast, face, penis enlargement, vagina reduction.

In a preferred embodiment the addition curable polysiloxane reactive system comprises a polysiloxane having ethylenically unsaturated moieties. The ethylenically unsaturated moiety or moieties can be present on an end Si, but also as a substituent to the polysiloxane. Vinyl and unsaturated aromatics such as phenyl are preferred examples of these moieties. Preferably an ethylenically unsaturated moiety terminated polysiloxane, more preferably a vinyl terminated polysiloxane such as vinyldialkyl terminated dimethyl polysiloxane, even more preferably a vinyldimethyl terminated dimethyl polysiloxane is used in the addition curable polysiloxane system. The above polysiloxanes are commercially available from several manufacturers including Nusil, Dow Corning and UCT.

The addition curable polysiloxane system also comprises a catalyst, in particular a precious metal catalyst, preferably Pt. More preferably this catalyst is present as an organic Pt(0) complex such as a Pt(0) vinyl siloxane complex.

The addition curable polysiloxane system also comprises a crosslinker for addition curing of the base polymeric polysiloxanes described above. Typically the crosslinker will be a hydrosilicone crosslinker having a number of hydrogen atoms capable of reaction with the ethylenically unsaturated groups of the base polymer(s). Preferably, the crosslinker comprises hydrogendialkyl terminated siloxy groups, preferably hydrogendimethyl terminated siloxy groups. Another preferred type of crosslinker is polyalkyl hydrosiloxanes wherein hydrogen is present in each unit of the siloxane chain. Polymethylhydrosiloxanes are an example. The crosslinker will hydrosilylate olefins in the presence of precious metal catalysts, such as Pt.

A system according to the invention is typically such that the viscosity thereof allows extrusion through a needle, e.g. an 11 gauge needle, and such that easy application is possible. Curing takes place at low temperature. During in vivo curing the local temperature will not rise higher than 40° C., in particular not higher than body temperature, so that no damage occurs.

Typically the addition curable polysiloxane system also comprises a tracer in order to monitor the progress of the treatment of the invention, in particular a material traceable by ultrasound as used in echoscopy and/or a radiopaque material for monitoring by X-rays. Suitable tracer materials include silver powder, barium sulphate, bismuth trioxide, zirconium dioxide, tantalum or titanium powders or fibers, calcium sulphate, calcium phosphate, hydroxyapatite, tri calcium phosphate, and other medically appropriate opacifier agents. A preferred tracer material is titanium dioxide, which can be traced very well using ultrasound and is also capable of being traced using X-rays. Furthermore this material does not settle in the base composition parts, contrary to silver powder and barium sulphate. It has also appeared that echoscopy suffices in a number of treatments to monitor the deposition sites of the addition curable polysiloxane system. This is beneficial as echoscopy is frequently experienced less heavy compared to X-ray examination. In a further preferred embodiment a bactericidal agent is also present in the composition. Silver powder is an example thereof. Preferably the bactericidal agent comprises an Ag⁺ zeolite, especially an Ag⁺ nano zeolite. Such a zeolite does not settle, allows ion exchange, e.g. substitution of silver ions by sodium ions from the body. This is a relatively slow process thereby effectively extending the time period during which Ag⁺ ions are released. Furthermore this bactericidal agent prevents the formation of a so called biofilm affecting adhesion and promoting inflammation risk. Ag⁺ zeolite has also radiopaque properties.

A non-resorbable filler, preferably a particulate filler, like silicate could also be present in the addition curable polysiloxane system according to the invention. Like the tracer material and bactericidal agent these filler particles are permanently encapsulated in the silicone polymer matrix upon curing.

The composition is advantageously packaged as a kit of parts, comprising at least a first container with a catalyst, and a second container with the cross-linking agent, wherein advantageously the base polysiloxane polymer(s) is/are present in both containers. A double barreled syringe is a preferred example of such a kit of parts.

In a further preferred embodiment the addition curable polysiloxane system is provided as a two component system, comprising as part A in a first container:
a polysiloxane having an ethylenically unsaturated moiety,
a catalyst,
optionally a tracer material, a bactericidal agent and/or a filler and as part B in a second container;
a polysiloxane having an ethylenically unsaturated moiety,
a crosslinker,
optionally a tracer material, a bactericidal agent and/or a filler.

A preferred embodiment of a two component dispensing device comprises a double barreled syringe having two parallel cylindrical compartments, each provided with a plunger, the ends thereof outside the compartments are coupled. The outlets of each compartment exit into a mutual outlet, which before actual use is preferably provided with a static mixer. A small diameter tube such as a catheter, or a needle assembly is attached to the static mixer.

For treating female urinary incontinence the composition is preferably deposited in the tissue surrounding the urethra at various circumferential positions, e.g. 3 positions rotated by 120° forming an interrupted annulus of cured material.

Instead of a static mixer a two component injection device including an internal mixer, as for example disclosed in PCT/NL2004/000827 and PCT/NL2005/000268, can be used. The preferred, advantageous and/or beneficial embodiments of the various components of the composition similarly apply to this embodiment.

In particular, the composition is packaged in a syringe having two containers temporarily sealed from each other, wherein a first part comprising the catalyst of the reactive system is present in a first container and a second part comprising the crosslinker of the reactive system is present in a second container. Preferably the first and second containers comprise, part A and part B as described above.

After filling the package and content are subjected to a sterilizing treatment. It has appeared that sterilizing using ethyleneoxide as a sterilizing agent is effective.

Advantageously the addition curable polysiloxane is applied in an effective amount for achieving a durometer of about 25-50 Shore A, a tensile strength of 400-800 psi, and elongation of 120-175%.

The invention also relates to a prepackaged addition curable polysiloxane system for use in soft tissue treatment in a living creature, in particular a human being, with a bulking material prepared in situ from a composition comprising an addition curable polysiloxane system, comprising a two component dispensing device having two containers separated from each other by a temporary seal, wherein a first container comprises as part A:
a polysiloxane having an ethylenically unsaturated moiety,
a catalyst,
optionally a tracer material, a bactericidal agent and/or a filler and as part B in a second container;
a polysiloxane having an ethylenically unsaturated moiety,
a crosslinker,
optionally a tracer material, a bactericidal agent and/or a filler.

The preferred, advantageous and/or beneficial embodiments of the various components of the composition similarly apply to this device. Advantageously this package also comprises a mixing means, preferably a static mixer, and/or at least one injection needle.

A further aspect of the invention concerns the use of an addition curable polysiloxane system in preparing a composition for use in soft tissue treatment in a living creature. The preferred, advantageous and/or beneficial embodiments of the various components of the composition similarly apply to this use according to the invention.

The invention also provides a method of treating soft tissue in a living creature, in particular treatment of incontinence, reflux, and in filling a cavity resulting from prostatectomy, reconstructive and cosmetic surgery, comprising the step of incorporating an addition curable polysiloxane composition comprising an ethylenically unsaturated moiety terminated polysiloxane, a crosslinker and a catalyst in effective amounts for allowing an addition curing in situ, in the respective soft tissue and allowing said composition to cure. The preferred, advantageous and/or beneficial embodiments of the various components of the composition similarly apply to this use according to the invention.

In treating incontinence the injectable composition is delivered in the tissue surrounding the urethra, e.g. at 3 positions spaced apart by 120°. In a preferred embodiment the material is applied at 4 positions, in particular at 2, 5, 7 and 10 hours, around the urethra. The amounts applied at each position may be equal, but can also differ, e.g. at 2 and 10 hours in the range of 0.5-0.8 ml and at 5 and 7 hours 0.8-1.4 ml.

Positioning of the injection needles and delivering the composition is monitored e.g. by a scope integrated in an applicator provided with plural carrier positions for the injection device, e.g. as indicated above.

Numerous characteristics and advantages have been set forth in the foregoing description, together with details of structure and function. While the invention has been disclosed in several forms, it will be apparent to those skilled in the art that many modifications, additions, and deletions, especially in matters of shape, size, and arrangement of parts, can be made therein without departing from the spirit and scope of the invention and its equivalents as set forth in the following claims. Therefore, other modifications or embodiments as may be suggested by the teachings herein are particularly reserved as they fall within the breadth and scope of the claims here appended.

What is claimed is:

1. A soft tissue treatment composition comprising an addition curable polysiloxane system.

2. The soft tissue treatment composition of claim 1, wherein the addition curable polysiloxane system comprises:
a polysiloxane;
a catalyst; and
a crosslinker.

3. The soft tissue treatment composition of claim 2, wherein the addition curable polysiloxane system further comprises one or more of a tracer material, a bactericidal agent and a filler.

4. The soft tissue treatment composition of claim 1, wherein the addition curable polysiloxane system comprises a polysiloxane having an ethylenically unsaturated moiety.

5. The soft tissue treatment composition of claim 1, wherein the addition curable polysiloxane system comprises an ethylenically unsaturated moiety terminated polysiloxane.

6. The soft tissue treatment composition of claim 1, wherein the addition curable polysiloxane system comprises vinyl terminated polysiloxane.

7. The soft tissue treatment composition of claim 6, wherein the vinyl terminated polysiloxane comprises vinyldialkyl terminated dimethyl polysiloxane.

8. The soft tissue treatment composition of claim 6, wherein the vinyl terminated polysiloxane comprises vinyldimethyl terminated polysiloxane.

9. The soft tissue treatment composition of claim 1, wherein the addition curable polysiloxane system comprises a Pt catalyst.

10. The soft tissue treatment composition of claim 1, wherein the addition curable polysiloxane system comprises a Pt(0) organic complex.

11. The soft tissue treatment composition of claim 1, wherein the addition curable polysiloxane system comprises a Pt(0) vinyl siloxane complex.

12. The soft tissue treatment composition of claim 1, wherein the addition curable polysiloxane system comprises a hydrosilicone crosslinker.

13. The soft tissue treatment composition of claim 12, wherein the hydrosilicone crosslinker comprises hydrogen dialkyl terminated siloxy groups.

14. The soft tissue treatment composition of claim 12, wherein the hydrosilicone crosslinker comprises hydrogen dimethyl terminated siloxy groups.

15. The soft tissue treatment composition of claim 1, wherein the addition curable polysiloxane system comprises a first container with a catalyst, and a second container with a crosslinker, wherein at least one of the first and the second container further comprise a polysiloxane.

16. A soft tissue treatment composition comprising an addition curable polysiloxane system, wherein the addition curable polysiloxane system comprises:
a polysiloxane;
a catalyst;
a crosslinker; and
optionally, one or more of:
a tracer material;
a bactericidal agent; and
a filler.

17. The soft tissue treatment composition of claim 16, wherein:
the polysiloxane comprises an ethylenically unsaturated moiety terminated polysiloxane;
the catalyst comprises a Pt(0) vinyl siloxane complex; and
the crosslinker comprises hydrogen dimethyl terminated siloxy groups.

18. The soft tissue treatment composition of claim 16 comprising a tracer material comprising titanium dioxide.

19. The soft tissue treatment composition of claim 16 comprising a bactericidal agent comprising Ag⁺ zeolite.

20. The soft tissue treatment composition of claim 16, wherein the addition curable polysiloxane system comprises a first container comprising:
a portion of the polysiloxane comprising a polysiloxane having an ethylenically unsaturated moiety; and
the catalyst; and
a second container comprising:
a portion of the polysiloxane comprising a polysiloxane having an ethylenically unsaturated moiety; and
the crosslinker.

21. The soft tissue treatment composition of claim 20, wherein the containers are parts of a two component dispensing device having the two containers separated from each other by a temporary seal.

22. The soft tissue treatment composition of claim 21, wherein the two component dispensing device further comprises a mixing means.

23. The soft tissue treatment composition of claim 22, wherein the mixing means comprises a static mixer.

24. The soft tissue treatment composition of claim 22, wherein the mixing means comprises at least one injection needle.

25. A method of treating soft tissue in a living creature comprising:
preparing a bulking agent in situ from the composition of claim 1; and
treating the soft tissue with the bulking agent.

26. The method according to claim 25, wherein treating the soft tissue with the bulking agent is selected from the group consisting of the treatment of: incontinence, reflux, reconstruction surgery, cosmetic surgery, tissue expansion, filling a body cavity caused by surgery, and prostatectomy.

27. The method according to claim 25, wherein preparing the bulking agent comprises:
applying the composition of claim 5;
applying a catalyst;
applying a crosslinker; and
curing the bulking agent, in situ, in proximity to the soft tissue.

28. The method according to claim 25 further comprising monitoring one or more of the preparing and treating steps.

29. The method according to claim 28, wherein monitoring one or more of the preparing and treating steps comprises monitoring via X-ray imaging.

30. The method according to claim 28, wherein monitoring one or more of the preparing and treating steps comprises monitoring via echoscopy.
